(19) United States
(12) Patent Application Publication
(10) Pub. No.: US 2003/0041481 A1
(43) Pub. Date: Mar. 6, 2003

(54) Orthotic footwear and insole thereof

(75) Inventors: J. Pat Evans, Dallas, TX (US); John G. Pearce, Fort Worth, TX (US); Duane E. Peoples, Allentown, PA (US)

Correspondence Address: SIDLEY AUSTIN BROWN & WOOD LLP, 717 NORTH HARWOOD, SUITE 3400, DALLAS, TX, 75201, US

(73) Assignee: BOOT ROYALTY COMPANY

(21) Appl. No.: 09/945,006

(22) Filed: Aug. 31, 2001

(51) Int. Cl. A43B 7/14; A61F 5/14

(52) U.S. Cl. 36/145; 36/168; 36/174; 36/180

(57) ABSTRACT

An insole for footwear comprises a footbed having an upper surface and a lower surface, with a raised arch support portion on the upper surface of the footbed capable of supporting a longitudinal arch and a transverse arch of a human foot. The shape of the insole can generally correspond to the shape of a plantar surface of the human foot, thereby providing support to substantially the entire plantar surface of the foot.

### FIELD OF THE INVENTION

In general, the present invention relates to orthotic footwear. In one aspect, the invention relates to footwear having an orthotic insole. In another aspect, the invention relates to an orthotic insole for footwear.

### BACKGROUND OF THE INVENTION

Orthotic insoles are used in many circumstances to reduce and relieve the pain and discomfort that many people experience due to chronic foot problems. Orthotic footwear devices and footwear incorporating orthotic insoles are often used to help prevent or to combat the effects of the various foot problems people may experience.

Many of the foot problems that a person may experience are due, at least in part, to the aging effects on a foot. Specifically, as a person ages, critical plantar ligaments in the foot gradually deteriorate to a weakened state. When functioning properly, these plantar ligaments hold the bones of the foot in a particular alignment, which forms the different arches of the foot. Generally speaking, a longitudinal arch of the foot is weight-bearing and thus helps facilitate the support function of the foot. A transverse arch, on the other hand, facilitates movement of the foot by helping to keep the plantar surface of the foot in contact with the ground despite the unevenness of the ground surface and by working in concert with the ankle joint to help propel the foot off the ground during the toe-off portion of human gait. Over time, however, the once resilient plantar ligaments that supported the bony longitudinal and transverse arches of the foot become stretched and/or less resilient, and consequently, the bones of the foot begin to splay, or spread apart. As a result, the arches of the foot that were once resiliently supported by the strength of the plantar ligaments “fall” to a more flattened position. As the plantar ligaments become weaker, problems associated with “fallen arches”progressively worsen and the splaying of the bones of the foot continues. Accordingly, attempts to relieve patient discomfort and to correct the problems associated with deterioration of the ligaments in the foot have produced many orthotic footwear devices, including orthotic insoles.

Many conventional insoles for footwear are provided with a longitudinal arch support feature intended to protect and support the longitudinal arch of a foot. This longitudinal arch support feature is usually in the form of a protrusion on a lower surface of an insole or on an upper surface of an insole. The protrusion, in general, is positioned at an area of the insole where the longitudinal arch portion of the plantar surface of the foot will be placed. Accordingly, when the insole is placed in a shoe, the protrusion on the surface of the insole is raised above the other surfaces of the insole, and will be substantially aligned with a longitudinal arch of the foot. The user is thus provided with an insole that is capable of supporting a longitudinal arch of the foot.

However, conventional insoles primarily provide support to only the longitudinal arch. Conventional insoles thus fail to provide other areas of the foot, for example the transverse arch, with sufficient support. Especially in a construction where the supportive protrusion of the insole is located on the lower side of the insole, other areas of the foot receive virtually no support from the conventional insoles.

Further, many conventional insoles are constructed of materials that have a low resistance to compression set. Over time and continued use, the repeated compression of such materials causes the conventional insoles to become thin and ineffective as the materials gradually become set in a compressed state. The amount of support provided by the insole thus decreases as the supportive structure of the insole deforms under the weight of continuous usage. As a result, the orthotic properties of the insole degrade since the foot is allowed to progressively return to a problematic position.

Rigid insoles are also available, but are usually customized on an individual basis. Due to the difficulty associated with creating an orthotic insole that provides comfort as well as treatment for many different patients with many different ailments, orthotic insoles are often created, or customized, to fit an individual, according to a specific ailment. These insole structures are usually rigid, and are used to properly align regions of the foot, and to hold the foot in an aligned position. Although these devices may effectively realign the foot, rigid orthotic structures do not provide adequate cushioning and impact absorption. Consequently, they do not provide much comfort for a user, especially when worn for extended periods.

Accordingly, there exists a need for an orthotic insole that provides adequate support for substantially the entire plantar surface of a foot, including both the longitudinal arch and the transverse arch. Further, there exists a need for an insole that resists compression set due to continuous usage and repeated compression. There is also a need for an insole that is capable of properly aligning the foot while providing proper cushioning and shock absorption. Further still, a need exists for an insole, with all of the above features, that is readily available without a custom-fitting requirement.

### BRIEF SUMMARY OF THE INVENTION

Accordingly, the present invention provides an orthotic insole for footwear and footwear incorporating an orthotic insole that is both comfortable to a user and effective in minimizing foot ailments associated with misalignment of the skeletal structure of the foot.

According to one aspect of the present invention, an insole for footwear includes a footbed having an upper surface and a lower surface, wherein the upper surface has a raised arch support portion including a significant longitudinal and transverse arch topography. Due to the topography of the raised arch support portion, the insole is capable of providing support to a transverse arch and a longitudinal arch of a human foot.

In another aspect of the invention, at least a portion of an upper surface of the footbed has a contour generally corresponding to that of a plantar surface of a human foot.

According to another aspect of the present invention, an insole for footwear includes a footbed having an upper surface and a lower surface, wherein the upper surface has a surface topography having a prominent arch feature whose dimensional characteristics and location on the insole are defined by known and identifiable anatomical landmarks of the foot such that the arch feature can provide support to a transverse arch and a longitudinal arch of a human foot.

In yet another aspect of the present invention, an insole for footwear includes an upper surface having a surface topography that includes a raised arch feature having an edge defined by the algorithmic points of tangency corresponding to the plantar topography of the transverse arch and the longitudinal arch of a human foot. In a still further aspect of the present invention, the edge of the raised arch feature starts at the medial edge of the insole at a medial point located posterior to the bony protuberance of the first metatarsal head, curves laterally and forwardly to a forwardmost point located midway between the second and third metatarsal heads, curves laterally and rearwardly across the fourth metatarsal head and along the medial edge of the fifth metatarsal shaft, curves medially and rearwardly across the cuboid, and ends at the medial edge of the insole at a rearwardmost medial point located posterior to the Astragalas. In a still further aspect of the present invention, the surface topography of the raised arch feature has a height dimension which can be varied depending on the unique topography of the plantar surface of a user's foot.

In another aspect of the invention, a peripheral contour of the raised arch support portion of the insole generally conforms to a longitudinal arch and a transverse arch of a plantar surface of a human foot.

According to another aspect of the invention, an insole for footwear having a hindfoot region and a forefoot region comprises a footbed having an upper surface and a lower surface, wherein the upper surface has a raised arch support portion with a peripheral contour beginning at a point on the medial edge of the footbed generally corresponding to a forwardmost point of the longitudinal arch on a medial edge of the foot, extending laterally to a point on the footbed located substantially midway between a second metatarsal head and a third metatarsal head, extending to a point on the footbed corresponding to a fourth metatarsal head, extending in a generally posterior direction substantially tangent to a fifth metatarsal shaft, extending medially across a point corresponding to a cuboid of the foot, and terminating at a point on the medial edge of the footbed generally corresponding to a rearmost point of the longitudinal arch on the medial edge of the foot.

According to another aspect of the invention, footwear is formed comprised of a sole joined to an upper footwear portion to define a foot-receiving chamber. The sole has an inner surface and an outer surface. The footwear further can include an insole positioned on the inner surface of the sole of the footwear. The insole has a hindfoot region and a forefoot region and comprises a footbed having an upper surface and a lower surface, wherein the upper surface of the footbed has a raised arch support portion having a peripheral contour generally conforming to a longitudinal arch and a transverse arch of a human foot. The insole further comprises a first shock-absorbing portion that is disposed at the hindfoot region of the footbed and a second shock-absorbing portion that is disposed at the forefoot region of the footbed corresponding to a position under metatarsus heads one through five

According to another aspect of the invention, a method for forming a footwear device comprises providing an orthotic insole for supporting a longitudinal arch and a transverse arch of a plantar surface of a human foot, wherein the insole comprises a raised arch support portion having a peripheral contour generally conforming to the longitudinal arch and the transverse arch of the plantar surface of the human foot, calculating a three-dimensional contour of a foot-receiving cavity of the footwear device corresponding to a foot of the predetermined foot size, adjusting a height of the three-dimensional contour of the foot-receiving cavity to accommodate a height of the orthotic insole, and forming the footwear device according to the adjusted three-dimensional contour of the foot-receiving cavity.

### BRIEF DESCRIPTION OF THE DRAWINGS

Other advantages and features of the invention will become more apparent with reference to the following detailed description of the presently preferred embodiment thereof in connection with the accompanying drawings, wherein like reference numbers have been applied to like elements, in which:

FIG. 1 is a perspective view of an upper surface of an insole according to an embodiment of the present invention;

FIG. 2 is a top view of a top surface of an insole according to the embodiment of FIG. 1, with a skeletal structure of a foot superimposed thereon;

FIG. 3 is a perspective view of a lower surface of an insole according to the embodiment of FIG. 1;

FIG. 4 is a medial side view of the insole according to the embodiment of FIG. 1;

FIG. 5 is a lateral side view of the insole according to the embodiment of FIG. 1;

FIG. 6 is an exploded view of an insole of an embodiment of the present invention, illustrating the layers of the insole; and

FIG. 7 is a perspective view of a footwear device according to an embodiment of the present invention, the footwear device containing an insole and the view having portions of the footwear device broken away.

### DETAILED DESCRIPTION OF THE INVENTION

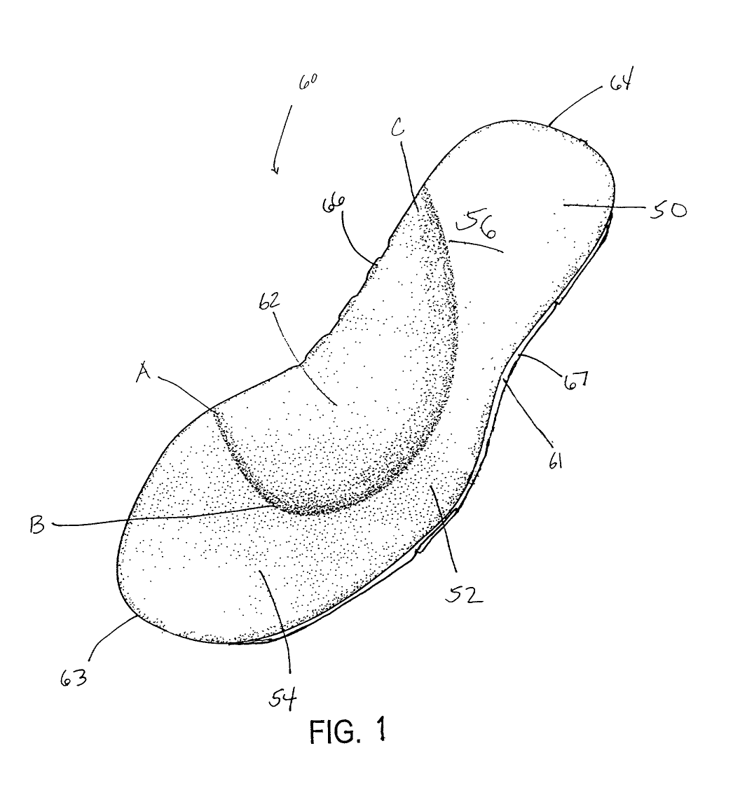

Referring now to the figures, FIG. 1 illustrates an insole 60 in accordance with one embodiment of the present invention. As shown, the shape of insole 60 generally corresponds to the shape of a human foot such that insole 60 is capable of supporting substantially the entire length and width of a plantar surface of a human foot.

Insole 60 includes a footbed 61 which is preferably made of a resilient material that allows insole 60 to be flexible for easy installation into footwear and for increased comfort while in use. Footbed 61 extends from a heel edge 64 to a toe edge 63 and from a medial edge 66 to a lateral edge 67 of insole 60, and has a peripheral contour generally corresponding to a peripheral contour of a plantar surface of a human foot. Footbed 61 includes three general regions: a hindfoot region 50 bounded by medial edge 66 and lateral edge 67 which extends from the heel edge 64, a forefoot region 54 bounded by medial edge 66 and lateral edge 67 which extends from the toe edge 63, and a midfoot region 52 bounded by medial edge 66 and lateral edge 67 which extends between the hindfoot region 50 and the forefoot region 54. Hindfoot region 50, midfoot region 52, and forefoot region 54 are defined herein for ease of reference to indicate general areas of the insole, and thus the exact boundaries and/or limits between such regions are not critical to the definition of the present invention.

As depicted in FIG. 1, an upper surface 56 of footbed 61 is preferably provided with an arch support portion 62, which is a region of footbed 61 that slopes upwardly and is raised relative to the other areas of upper surface 56. Arch support portion 62 is positioned on the upper surface 56 of footbed 61 such that it can provide support for a transverse arch and a longitudinal arch of the foot. The peripheral contour of arch support portion 62 begins at position A located at the medial edge 66 of footbed 61 in forefoot region 54 and follows a curve that extends through midfoot region 52 and terminates at position C located at the medial edge 66 of footbed 61 in hindfoot region 50. More specifically, the curve of arch support portion 62 begins at position A and extends laterally toward the lateral edge 67 of footbed 61, as well as forwardly toward the toe edge 63 of footbed 61. Upon reaching a forwardmost position B on the upper surface 56 of footbed 61, arch support portion 62 begins a rearward curve toward the lateral edge 67 of footbed 61, without encountering the lateral edge 67 of insole 60. Thereafter, the peripheral contour of arch support portion 62 extends back toward the medial edge 66 as the radius of curvature gradually decreases until arch support portion 62 ultimately terminates at a rearmost position C at the medial edge 66 of footbed 61. Although the peripheral contour of arch support portion 62 is described generally as a continuous curve, the peripheral contour of arch support portion 62 can be formed by other suitable contours, including curved or straight line segments connecting the disclosed anatomical points of reference associated with the longitudinal and transverse arches.

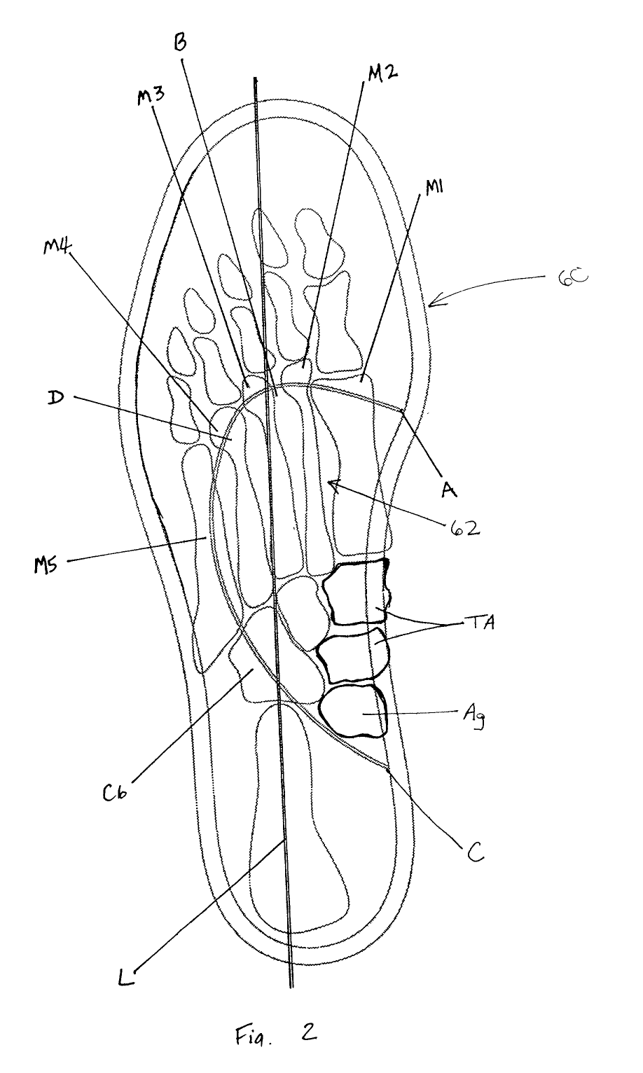

According to the description given in the embodiment described above in conjunction with FIG. 1, the peripheral contour of arch support portion 62 generally follows the shape of a longitudinal arch and a transverse arch of a human foot. More specifically, when an insole of a predetermined size is positioned such that an upper surface of the insole is facing and aligned with a plantar surface of a human foot having a size corresponding to the predetermined size of the insole, the insole conforms to the plantar topography of the human foot and is capable of providing support for both the longitudinal arch and the transverse arch of the foot. This feature will be explained in more detail with reference to FIG. 2.

FIG. 2 is an illustration of upper surface 56 of an insole of the present embodiment, including an outline of the peripheral contour of arch support portion 62, with a partial skeletal structure of a human foot superimposed thereon. The area of upper surface 56 contained within this outline is raised relative to the other areas of upper surface 56, thus forming arch support portion 62. As can be seen in FIG. 2, arch support portion 62 begins at position A, which corresponds to a forwardmost medial point of the longitudinal arch of a human foot, posterior to the bony protuberance of a first metatarsal head M1. The peripheral contour of arch support portion 62 then extends laterally and forwardly to a position B on the upper surface 56 of the insole defined anatomically as an area located midway between a second metatarsal head M2 and a third metatarsal head M3, at which point the forwardmost lateral radius of the transverse arch has been created (position B is forward of position A because the second and third metatarsals are usually slightly longer than the first metatarsal). Following the general curve of the transverse and longitudinal arches, the peripheral contour then curves laterally and rearwardly to a position D, which is associated with a position of the fourth metatarsal head M4. Thereafter, the peripheral contour continues to curve laterally and rearwardly until it reaches the medial edge of the fifth metatarsal shaft M5. Without extending to a point lateral of fifth metatarsal shaft M5, the peripheral contour of arch support portion 62 then follows a gradual medial and rearward curve that generally follows the full length of fifth metatarsal shaft M5 tangentially. After the peripheral contour extends tangentially along fifth metatarsal shaft M5, its curve radiuses proximally to an imaginary midline L of the upper surface 56 of the insole and traverses a position on upper surface 56 generally corresponding to the anatomical position of a cuboid Cb. Finally, the peripheral contour of arch support portion 62 terminates at a position C located at the medial edge 66 of footbed 61 rearward of position A which is posterior to the Astragalas Ag of the foot.

While support portion 62 is shown in the preferred embodiment of FIG. 1 as positioned on the upper surface 56 at the insole, it will be appreciated that the invention can also be practiced by placing support portion 62 on the lower surface of the insole.

According to the above-described geometry of insole 60 and arch support portion 62 with respect to FIG. 1 and FIG. 2, an upper surface 56 of footbed 61 conforms to the plantar topography of a human foot and is thus capable of providing support to substantially the entire plantar surface of the human foot. Moreover, arch support portion 62 is positioned to extend and support substantially the full and complete length of the longitudinal arch of the foot while also supporting the lateral extension across the transverse arch of the foot.

Many conventional insoles primarily provide support for the longitudinal arch of the foot while failing to provide sufficient support to the transverse arch of the foot. However, proper support of the transverse arch of the foot is beneficial when attempting to properly align and support the foot. Thus, in accordance with the described embodiment of the insole of the present invention, the raised structure of arch support portion 62 is positioned both longitudinally along the medial edge 66 of insole 60 and laterally to a position lateral of the midline L of footbed 61 such that the longitudinal arch as well as the transverse arch of the foot are well supported. Accordingly, unlike conventional insoles, arch support portion 62 is capable of providing support for the transverse arch of the foot by increasing the total surface area of the upper surface 56 of insole 60 to more closely follow the plantar surface of the foot. By increasing the area of the upper surface 56 of insole 60 that more closely follows the plantar surface of the human foot, the device effectively reduces downward pressures created when the weight of the wearer is fully applied to the insole. In other words, the insole of the present invention distributes pressure over a greater area, thus resulting in lower forces experienced at discrete points on the plantar surface of the foot. This effect may also be defined by the equation: Pressure=Force÷Area.

Due to the location and geometry of arch support portion 62 of insole 60, support is provided to a portion of the transverse arch that forces the foot into a properly aligned position. That is, pressure from insole 60 is applied upwardly to portions of the transverse and longitudinal arches of the foot near the necks and heads of the metatarsals, and the upward pressure forces the foot into an alignment that draws the bones of the foot, namely the metatarsals, back to a “normal” position. In this way, insole 60 functions as an artificial arch, which can bring the metatarsal arch into proper alignment, even in a case when the plantar ligaments are no longer resilient, and the arches of the foot have “fallen.”

The height or thickness of arch support portion 62 of insole 60 at various points along its surface topography is not critical to this invention. However, in a particularly preferred embodiment, the height dimension of arch support portion 62 reaches a maximum at a point located substantially midway between first metatarsal head M1 and the Astragalas Ag of the foot, which point generally corresponds to a location between the Tibialis Anticus TA. In an even more preferred embodiment, the maximum height dimension is approximately 0.375 inches high relative to the plane of the upper surface 56 of insole 60. Again, however, the particular height or thickness of arch support portion 62 can be varied without departing from the scope of this invention.

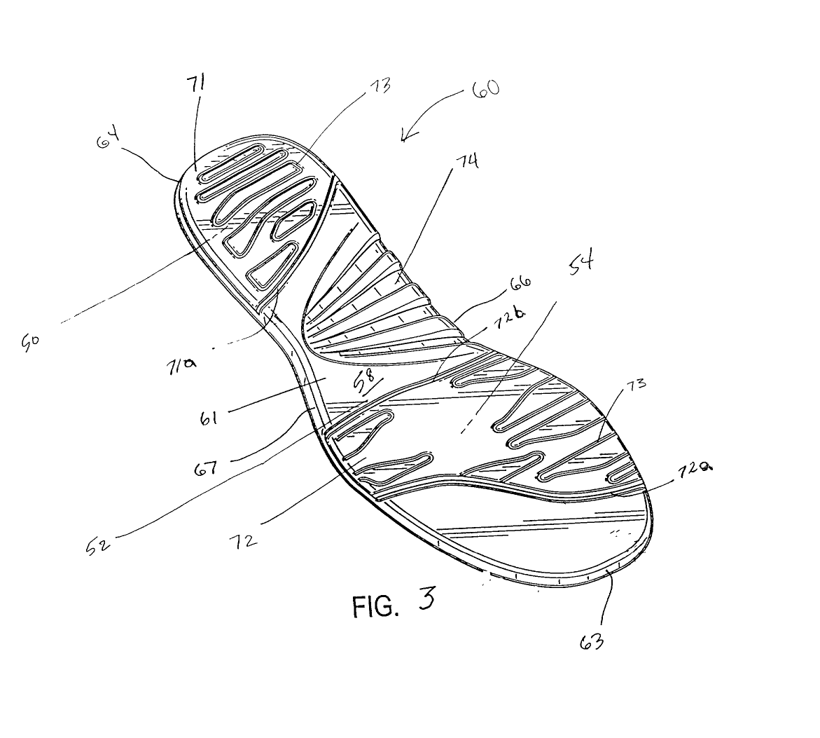

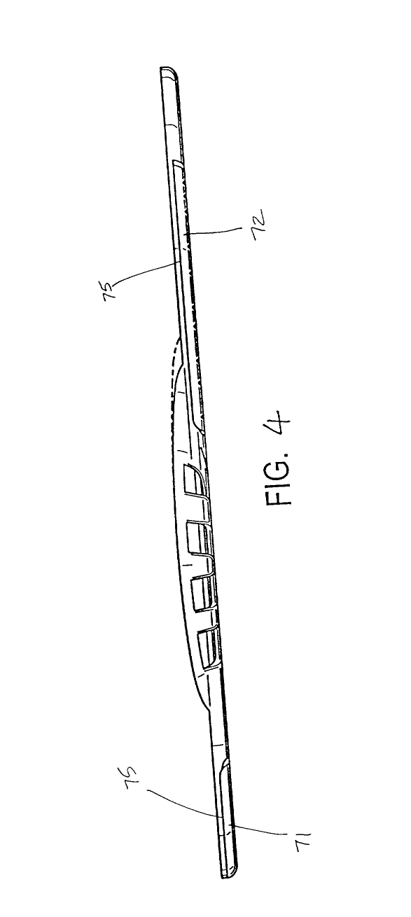

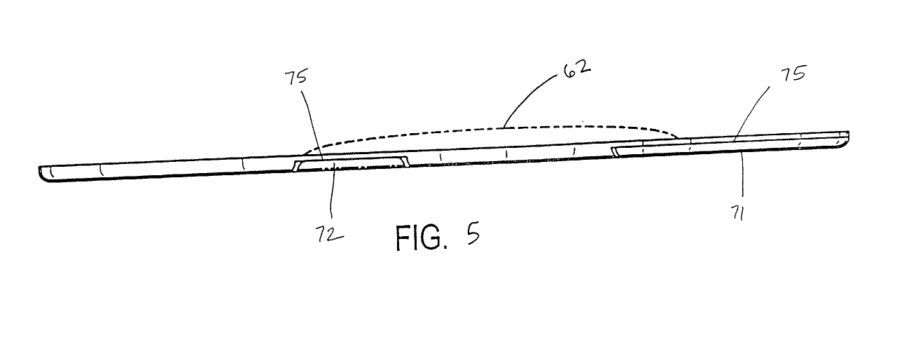

FIG. 3 illustrates a lower surface 58 of insole 60, according to one embodiment of the present invention. On the lower surface 58 of insole 60, footbed 61 is provided with a first shock-absorbing portion 71 and a second shock-absorbing portion 72. According to FIG. 3, footbed 61 is provided with first shock-absorbing portion 71 generally positioned in the hindfoot region 50 and with second shock-absorbing portion 72 generally positioned in the forefoot region 54 of insole 60. As can be seen in FIGS. 4 and 5, which illustrate views of the medial edge 66 and the lateral edge 67, respectively, of insole 60, each shock-absorbing portion 71, 72 can be positioned in recessed portions 75 in footbed 61 of insole 60.

In one embodiment, first shock-absorbing portion 71 is provided beginning at the heel edge 64 of footbed 61 and extending forwardly toward the midfoot region 52 of footbed 61 to the boundary line 71 a. First shock-absorbing portion 71 is provided at the hindfoot region 50 of the insole to absorb an impact when such region strikes a surface, and generally corresponds to the shape of the heel of the foot. According to the preferred embodiment illustrated in FIG. 3, first shock-absorbing portion 71 terminates at a point on the lateral edge 67 of footbed 61 which is more forward on insole 60 than the termination point of first shock-absorbing portion 71 on the medial edge 66. Accordingly, first shock-absorbing portion 71 provides more support to a lateral side of the heel than to the medial side of the heel since the lateral side of the heel generally experiences a greater impact force at the moment of heel strike. As can be understood, however, the shape of first shock-absorbing portion 71 is not critical to the invention and thus first shock-absorbing portion 71 can have any shape which provides support and absorbs impact forces during human gait.

Second shock-absorbing portion 72 of insole 60 is preferably provided at a forefoot region 54 on the lower surface 58 of footbed 61 between front boundary line 72a and rear boundary line 72b. Second shock-absorbing portion 72 is generally positioned to correspond to the position of the metatarsal necks and heads of a foot when the foot is seated on insole 60. In the preferred embodiment illustrated in FIG. 3, second shock-absorbing portion 72 does not extend all the way to toe edge 63; instead, the forwardmost point of second shock-absorbing portion 72 begins at a point on the medial edge 66 of insole 60 forward of the position of first metatarsal head M1 and then curves laterally and rearwardly across insole 60 to a point on the lateral edge 67 such that second shock-absorbing portion 72 underlies and can support each of the five metatarsal heads. As will be understood, any other arrangement or shape can be used which provides support and absorbs impact to the toes and metatarsal heads during human gait.

According to the above-described embodiment, each of first shock-absorbing portion 71 and second shock-absorbing portion 72 can be provided to protect the portions of the foot that exert and receive the greatest amount of force generated through the stages of human gait. This can be particularly beneficial considering the force or impact during “heel strike” can often be fives times body weight, followed by a secondary force or impact just prior to “toe-off” that results when the metatarsal heads collide with the ground during the subsequent weight transfer phase of gait. More specifically, first shock-absorbing portion 71 is disposed in the hindfoot region 50 of insole 60 to provide support and impact absorption for the heel area of the foot, and second shock-absorbing portion 72 is disposed in the forefoot region 54 of insole 60 to provide support and impact absorption to the forward portions of the metatarsals.

Additionally, each of first shock-absorbing portion 71 and second shock-absorbing portion 72 can be provided with deformation grooves 73, or other depressions or indentations of any suitable configuration, throughout the lower surface of the material that forms each of first shock-absorbing portion 71 and second shock-absorbing portion 72. These grooves 73 allow material deformation, thus permitting the shock attenuating material to displace within an area defined by its provided geometry when placed under load bearing conditions. More specifically, the material used in first shock-absorbing portion 71 and second shock-absorbing portion 72 reduces the speed or velocity at which the foot strikes the ground by using a viscoelastic material, and the force attenuating characteristics of this material are enhanced by the inclusion of deformation geometry. By effectively slowing the rate at which a foot strikes the ground, the resulting impact forces are also reduced, as can be defined by the equation: Force=Mass×Acceleration.

For example, at a time of heel strike, first shock-absorbing portion 71 receives an impact force when the heel of a footwear device containing insole 60 strikes a surface. At this time, first shock-absorbing portion 71 will deform according to the position of the received impact. Instead of transmitting the impact force from heel strike to the foot of a wearer, deformation grooves 73 of first shock-absorbing portion 71 allow the shock-absorbing material to be deformed during the force of impact. Thus, a force from a heel strike on a surface is at least partially attenuated before the force reaches the foot of a user. Similarly, at a time of forefoot push-off prior to toe-off when the metatarsals, especially the first and second, are functioning to propel a person forward, second shock-absorbing portion 72 at least partially attenuates the impact force in the same way. The user therefore experiences less discomfort due to forces induced from surface contact during activity.

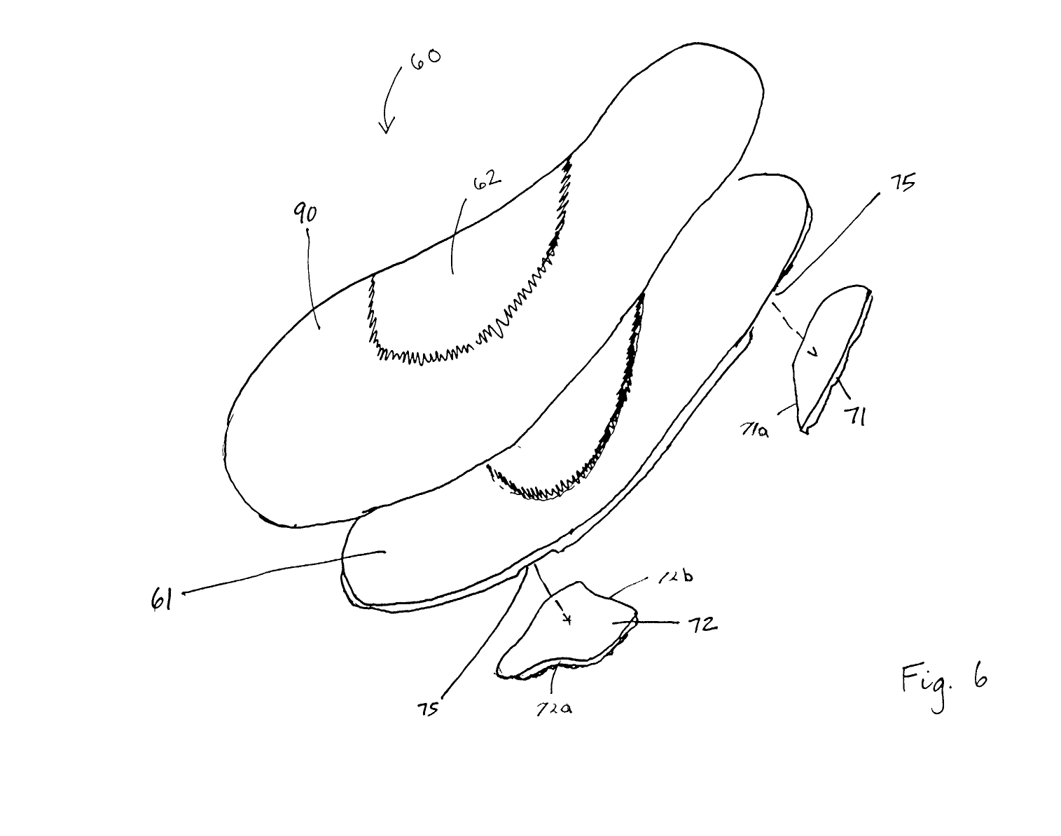

Insole 60 of the present invention can be formed of different materials. FIG. 6 illustrates a preferred construction of insole 60 where different portions of insole 60 are shown in an exploded view. The layers of materials forming the insole can be combined by any suitable method, which may include chemical cross polymerization, mechanical cementing, radio frequency welding, insert molding, or any other method known in the art.

While the present invention is not limited to any particular materials, the material that forms footbed 61 of insole 60 is preferably a soft and flexible material, and more preferably a soft, flexible foam material. Preferred flexible foam material includes a foam material which can be an open-celled polyurethane, having a molded density in a range of about 0.15 g/cc to about 0.45 g/cc cast, and a hardness ranging from about 50 Shore ‘000’ to about 30 Shore ‘A’. In a more preferred embodiment, the molded density of the foam material is in the range of about 0.25 g/cc to about 0.35 g/cc, and the hardness of the foam material can be in the range of about 20 Shore ‘00’ to 80 Shore ‘00’. Such open-celled polyurethane foam material also preferably has a Ball Rebound value of about 10 percent to 30 percent as defined by ASTM D-3574 (Test H). According to the above description, the foam material that forms footbed 61 adequately resists a compression set, and preferably has a compression set value that is less than 10 percent according to ASTM D 3574 (test D), and thus provides comfort and support to the foot of a wearer, even when worn for extended periods. As will be understood, the open-celled material described with reference to footbed 61 may be partially formed of close-celled material.

In a preferred embodiment, first shock-absorbing portion 71 and second shock-absorbing portion 72 can be made of a viscoelastic material. The viscoelastic material may be a viscoelastic polyurethane material which is preferably capable of providing a shock barrier between the primary weight bearing areas of the hindfoot and forefoot regions 50, 54 of insole 60. Any such material exhibiting viscoelastic properties, and which are capable of receiving and absorbing a force from an impact can be used in the shock-absorbing portions 71, 72. An example is Axidyne®, a viscoelastic polyurethane material marketed by Polymer Dynamics, Inc. The Axidyne® polymer provides excellent shock attenuation characteristics and thus lessens the forces transmitted to the foot from the impact of a foot contacting and traversing a surface. However, it will be understood that other materials capable of carrying out such shock absorbing capabilities known or to be developed may be used for forming the shock-absorbing portions 71, 72 without departing from the scope of this invention.

As shown in FIG. 6, a particularly preferred embodiment of insole 60 further includes a surface layer 90, which is preferably a textile lining that may be combined with insole 60 during a molding process. A number of textile materials can be used to overlay the upper surface 56 of footbed 61. Preferably, the surface layer 90 will provide good resistance to abrasion and improved comfort to a user. This lining material may be of varying constructions, including but not limited to blends of polyester and nylon fibers intended to promote preferred physical characteristic such as resistance to abrasion, pilling and friction. Additionally, these lining textiles may include such characteristics as moisture management treatments or additives, and anti-microbial treatments that are either inherent to the construction of the material or available as an additive.

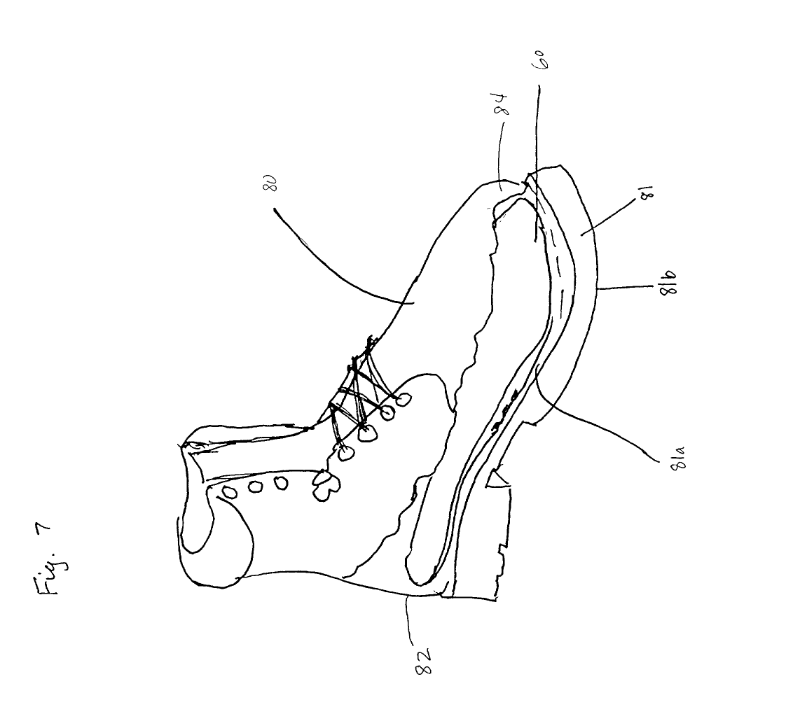

FIG. 7 illustrates an insole 60 of the present invention disposed on an inner surface 81a of a sole 81 of a footwear 80. Sole 81 of footwear 80 is also provided with an outer surface 81b which is generally beneath inner surface 81a and which comes into contact with a surface. As can be seen in FIG. 7, the lower surface of insole 60 conforms and aligns with the inner surface 81a of footwear 80. As insole 60 is constructed to support substantially an entire plantar surface of the foot, insole 60 extends substantially from a heel wall 82 of footwear 80 to a toe wall 84 of footwear 80. In addition, first shock-absorbing portion 71 and second shock-absorbing portion 72 can be provided on the lower surface of insole 60 (as shown in FIGS. 3-6) such that they abut the inner surface 81a of footwear 80 to provide additional support and shock absorption. Accordingly, during heel strike and forefoot push off, the primary weight bearing areas of the foot that absorb most of the impact are supported by the combination of the soft, flexible footbed 61 and the first and second shock-absorbing portions 71, 72. Moreover, deformation grooves 73 or the like can be provided in the shock-absorbing portions 71, 72 to help reduce the forces transmitted to the foot of a wearer by allowing deformation of insole 60 by the forces being absorbed.

The foregoing explanation with reference to FIG. 7 describes an embodiment of the present invention where insole 60 can be inserted into any footwear device to provide support, to assist in properly aligning the foot of a user, and to absorb impact forces occurring during human gait. In one aspect of the present invention, the footwear device is provided with sufficient volume or space within a foot-receiving cavity thereof to comfortably accommodate an insole and the foot of a wearer. More specifically, the foot-receiving cavity of the footwear device is constructed such that the size of the foot-receiving cavity is capable of retaining both an insole and a foot of a user having a predetermined foot size.

Because the insole of the present invention may have different dimensions from an insole not embodying the present invention, it may be desirable to use a footwear device with a foot-receiving cavity therein specifically designed to accommodate both the dimensions of the disclosed insole and the foot of a user. For instance, the foot-receiving cavity can preferably be sized to accommodate the length, width, height and other pertinent dimensions of the insole that will be placed in the footwear device. In addition, the three-dimensional size of the foot-receiving cavity of the footwear device can preferably be predetermined to correspond to a particular standard (or non-standard) foot size. Accordingly, a footwear device can be manufactured according to the specifications of a foot with a predetermined size such that an insole according to the present invention and a user's foot of the predetermined size can be comfortably accommodated without the need to make any additional modifications of the footwear device..

Although the present invention has been described with reference to a presently preferred embodiment, it will be appreciated by those skilled in the art that various modifications, alternatives, variations, and substitution of parts and elements, may be made without departing from the spirit of the invention. For example, although the embodiment described illustrates an insole that is placed into a boot with laces, it is understood that the insole is suitable for any variation of footwear. Similarly, if shock-absorbing portions are used, they may be aligned or shaped differently, or can be positioned in substantially any position of the insole to support and absorb impact at one or more of the primary weight bearing portions of the foot Furthermore, the present invention can take the form of a separate insole to be inserted into a footwear device, a standard footwear device having an insole inserted therein, a custom-fitted footwear device having an insole inserted therein, or an integral footwear device having an integrated insole. Thus, the present application is intended to cover such modifications, alternatives, variations and elements as fall within the scope of the appended claims.

What is claimed is:

1. An insole for supporting a longitudinal arch and a transverse arch of a plantar surface of a human foot, the insole comprising:
a raised arch support portion having a peripheral contour generally conforming to the longitudinal arch and the transverse arch of the plantar surface of the human foot.

2. An insole in accordance with claim 1, wherein the peripheral contour of the raised arch support portion is defined by a curve having a first endpoint located on a medial edge of the insole generally corresponding to a forwardmost medial point of the longitudinal arch and a second endpoint located rearward of the first endpoint on a medial edge of the insole,
wherein the curve extends forwardly and laterally from the first endpoint through a point generally corresponding to a midway point between a second metatarsal head and a third metatarsal head of the foot, extends rearwardly along a medial edge of a fifth metatarsal of the foot, and extends rearwardly and medially to the second endpoint.

3. An insole in accordance with claim 2, wherein the curve extends across a point generally corresponding to a cuboid of the foot as it extends rearwardly and medially from the fifth metatarsal of the foot to the second endpoint posterior to an Astragalas of the foot.

4. An insole in accordance with claim 1, wherein the peripheral contour of the raised arch support portion is defined by a curve having a first endpoint located on a medial edge of the insole generally corresponding to a forwardmost medial point of the longitudinal arch and a second endpoint located rearward of the first endpoint on a medial edge of the insole,
wherein the curve extends from the first endpoint, through a point generally corresponding to a first metatarsal head of the foot, through a point generally corresponding to a second metatarsal head of the foot, through a point generally corresponding to a third metatarsal head of the foot, through a point generally corresponding to a fourth metatarsal head of the foot, along an arc substantially tangent to a fifth metatarsal shaft, through a point generally corresponding to a cuboid of the foot, and to the second endpoint.

5. An insole in accordance with claim 1, further comprising a footbed having a peripheral contour generally conforming to a peripheral contour of the plantar surface of the human foot, wherein the raised arch support portion forms part of the footbed.

6. An insole in accordance with claim 1, wherein the raised arch support portion has a maximum height dimension substantially midway between a first metatarsal head and an Astragalas of the human foot.

7. An insole in accordance with claim 6, wherein the maximum height dimension of the raised arch support portion is approximately 0.375 inches greater than a height dimension of the insole.

8. An insole in accordance with claim 1, wherein the raised arch support portion has a maximum height dimension substantially midway between a Tibialis Anticus of the human foot.

9. An insole for supporting a longitudinal arch and a transverse arch of a plantar surface of a human foot, the insole comprising:
a footbed having an upper surface and a lower surface, the upper surface adapted to mate with the plantar surface of the human foot,
wherein the upper surface of the footbed comprises a raised arch support portion having a significant longitudinal and transverse arch topography bounded by a medial edge of the footbed and a peripheral contour beginning at a point on the medial edge of the footbed generally corresponding to a forwardmost point of the longitudinal arch on a medial edge of the foot, extending laterally to a point on the footbed located substantially midway between a second metatarsal head and a third metatarsal head, extending to a point on the footbed generally corresponding to a fourth metatarsal head, extending in a generally posterior direction substantially tangent to a fifth metatarsal shaft, extending medially across a point generally corresponding to a cuboid of the foot, and terminating at a point on the medial edge of the footbed generally corresponding to a rearmost point of the longitudinal arch on the medial edge of the foot.

10. An insole in accordance with claim 9, wherein the insole has dimensions generally corresponding to a human foot of a predetermined size.

11. An insole in accordance with claim 9, wherein the raised arch support portion has a maximum height dimension relative to the upper surface of the footbed substantially midway between a first metatarsal head and an Astragalas of the human foot.

12. An insole in accordance with claim 11, wherein the maximum height dimension of the raised arch support portion is approximately 0.375 inches high relative to a height of the upper surface of the footbed.

13. An insole in accordance with claim 9, wherein the peripheral contour of the raised arch support portion is curvilinear from the beginning point on the medial edge of the footbed to the terminating point on the medial edge of the footbed.

14. An insole in accordance with claim 9, wherein a peripheral contour of the footbed generally follows a peripheral contour of the plantar surface of the human foot such that the insole is capable of supporting substantially all of the plantar surface of the human foot.

15. An insole in accordance with claim 9, further comprising a surface layer provided on the upper surface of the footbed.

16. An insole in accordance with claim 15, wherein the surface layer is formed of a textile material.

17. An insole in accordance with claim 9, wherein the footbed has a hindfoot region and a forefoot region and is made of a flexible material.

18. An insole in accordance with claim 17, further comprising at least one shock-absorbing portion located at the hindfoot region of the footbed and at least one shock-absorbing portion located at the forefoot region of the footbed.

19. An insole in accordance with claim 18, wherein the lower surface of the footbed is provided with at least one recess in the hindfoot region and at least one recess in the forefoot region in which each of the at least one shock-absorbing portions can be disposed.

20. An insole in accordance with claim 19, wherein each of the at least one shock-absorbing portions has a plurality of grooves formed therein to accommodate deformation of the at least one shock-absorbing portion when a load is applied thereto.

21. An insole in accordance with claim 17, further comprising at least one shock-absorbing portion made of a viscoelastic material.

22. An insole in accordance with claim 21, wherein the footbed is made of an open-celled polyurethane material and the at least one shock-absorbing portion is made of a solid polyurethane material.

23. An insole in accordance with claim 22, wherein the open-celled polyurethane material of the footbed has a hardness value within a range from about 50 Shore ‘000’ to about 30 Shore ‘A’.

24. An insole in accordance with claim 22, wherein the open-celled polyurethane material of the footbed has a hardness value within a range from about 30 Shore ‘00’ to about 80 Shore ‘OO’.

25. An insole in accordance with claim 21, wherein the lower surface of the footbed is provided with at least one recess in which the at least one shock-absorbing portion can be disposed.

26. An insole in accordance with claim 25, wherein the at least one shock-absorbing portion has a plurality of grooves formed therein to accommodate deformation of the at least one shock-absorbing portion when a load is applied thereto.

27. An insole having a hindfoot region and a forefoot region, the insole comprising:
a footbed extending from the hindfoot region to the forefoot region and having an upper surface and a lower surface, the upper surface having a raised arch support portion having a peripheral contour generally conforming to a longitudinal arch and a transverse arch of a human foot;
a first shock-absorbing portion disposed at the hindfoot region of the footbed; and
a second shock-absorbing portion disposed at the forefoot region of the footbed.

28. An insole in accordance with claim 27, wherein the insole has dimensions generally corresponding to a human foot of a predetermined size.

29. An insole in accordance with claim 27, wherein the peripheral contour of the raised arch support portion begins at a point on a medial edge of the footbed generally corresponding to a forwardmost point of the longitudinal arch on a medial edge of the foot, extends laterally to a point on the footbed located substantially midway between a second metatarsal head and a third metatarsal head, extends to a point on the footbed generally corresponding to a fourth metatarsal head, extends in a generally posterior direction substantially tangent to a fifth metatarsal shaft, extends medially across a point generally corresponding to a cuboid of the foot, and terminates at a point on the medial edge of the footbed generally corresponding to a rearmost point of the longitudinal arch on the medial edge of the foot.

30. An insole in accordance with claim 27, wherein the lower surface of the footbed is provided with a recess in the hindfoot region and a recess in the forefoot region such that the first shock-absorbing portion can be positioned in the hindfoot recess and the second shock-absorbing portion can be positioned in the forefoot recess.

31. An insole in accordance with claim 27, further comprising a textile material layer on the upper surface of the footbed.

32. An insole in accordance with claim 27, wherein each of the shock-absorbing portions has a plurality of grooves formed therein for allowing deformation of the shock-absorbing portion when a load is applied thereto.

33. A footwear device comprising:
a sole having an inner surface and an outer surface;
a footwear upper portion attached to the sole, the footwear upper portion and the inner surface of the sole forming a foot-receiving cavity; and
an insole positioned on the inner surface of the sole, the insole comprising a footbed extending from a hindfoot region to a forefoot region and having an upper surface and a lower surface, the upper surface having a raised arch support portion having a peripheral contour generally conforming to a longitudinal arch and a transverse arch of a plantar surface of a human foot;
a first shock-absorbing portion disposed at the hindfoot region of the footbed; and
a second shock-absorbing portion disposed at the forefoot region of the footbed.

34. A footwear device in accordance with claim 33, wherein the peripheral contour of the raised arch support portion begins at a point on a medial edge of the footbed generally corresponding to a forwardmost point of the longitudinal arch on a medial edge of the foot, extends laterally to a point on the footbed located substantially midway between a second metatarsal head and a third metatarsal head, extends to a point on the footbed generally corresponding to a fourth metatarsal head, extends in a generally posterior direction substantially tangent to a fifth metatarsal shaft, extends medially across a point generally corresponding to a cuboid of the foot, and terminates at a point on the medial edge of the footbed generally corresponding to a rearmost point of the longitudinal arch on the medial edge of the foot.

35. A footwear device in accordance with claim 33, wherein the insole extends substantially from a heel wall of the footwear upper portion to a toe wall of the footwear upper portion and from a medial wall of the footwear upper portion to a lateral wall of the footwear upper portion such that the insole is capable of supporting substantially all of the plantar surface of the human foot.

36. A footwear device in accordance with claim 33, wherein the lower surface of the footbed is provided with recesses at the hindfoot region of the footbed and the forefoot region of the footbed, the first shock-absorbing portion being positioned in the recess at the hindfoot region and the second shock-absorbing portion being positioned in the recess at the forefoot region.

37. A footwear device in accordance with claim 33, further comprising a textile material layer on the upper surface of the footbed.

38. A footwear device in accordance with claim 33, wherein each of the first and second shock-absorbing portions has a plurality of voids formed therein adapted to accommodate material deformation of the shock-absorbing portion when a load is applied thereto.

39. A footwear device in accordance with claim 33, wherein the foot-receiving cavity is a partially enclosed compartment having dimensions substantially corresponding to a human foot of a predetermined size.

40. A footwear device in accordance with claim 39, wherein the insole is sized to generally correspond with the dimensions of the foot-receiving cavity and the predetermined size of the human foot.

41. A method for forming a footwear device having a foot-receiving cavity adapted to retain an orthotic insole corresponding to a predetermined foot size, the footwear device comprising:
providing an orthotic insole for supporting a longitudinal arch and a transverse arch of a plantar surface of a human foot, the insole comprising a raised arch support portion having a peripheral contour generally conforming to the longitudinal arch and the transverse arch of the plantar surface of the human foot;
calculating a three-dimensional contour of a foot-receiving cavity of the footwear device corresponding to a foot of the predetermined foot size;
adjusting a height of the three-dimensional contour of the foot-receiving cavity to accommodate a height of the orthotic insole; and
forming the footwear device according to the adjusted three-dimensional contour of the foot-receiving cavity.
